(19) United States
(12) Patent Application Publication
(10) Pub. No.: US 2010/0249595 A1
(43) Pub. Date: Sep. 30, 2010

(54) SYSTEM AND METHOD FOR AUTOMATIC CALIBRATION OF TRACKED ULTRASOUND

(75) Inventors: Sheng Xu, Rockville, MD (US); Jochen Kruecker, Washington Dc, DC (US)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/740,352

(22) PCT Filed: Nov. 5, 2008
(86) PCT No.: PCT/IB08/54619

§ 371 Date: Apr. 29, 2010

Related U.S. Application Data
(60) Provisional application No. 60/987,809, filed on Nov. 14, 2007.
(60) Provisional application No. 61/035,784, filed on Mar. 12, 2008.

(51) Int. Cl. A61B 8/14; G06K 9/62

(52) U.S. Cl. 600/443; 382/131

(57) ABSTRACT

A system and method of tracking ultrasound transducers or probes (12) with spatial localizers (16) achieves automatic calibration with a minimum addition of hardware to that required by earlier systems. An image-based tracking algorithm localizes control points in an image space (I). An unlimited number of points can then be used for ultrasound calibration, allowing for high calibration accuracy. The proposed calibration system (10) is simple and low cost. The calibration is fast and can be carried out automatically.

This application claims benefit of earlier filed provisional application entitled “SYSTEM AND METHOD FOR AUTOMATIC CALIBRATION OF TRACKED ULTRASOUND” to Sheng Xu et al., Ser. No. 60/987,809, filed Nov. 14, 2007 and assigned to the assignee of the present invention.

The present embodiments relate generally to medical systems and more particularly, to a method and apparatus for automatic calibration of tracked ultrasound.

Tracking ultrasound probes with spatial localizers has applications in surgical and interventional navigation, e.g., for fusion of the live ultrasound image with other image modalities. Calibration of the tracked ultrasound, i.e., determining the spatial relationship between the live ultrasound image and the attached spatial tracker, is necessary to enable these applications. Many traditional calibration methods are time consuming since they need human interaction to identify control points in ultrasound images. It is difficult for these manual methods to achieve high accuracy because that would require a high number of manually obtained control points. Although some recent calibration methods can perform the calibration automatically, they disadvantageously rely on complicated phantoms.

Using ultrasound for surgical navigation requires tracking the transducer in a global coordinate system. An optical or electromagnetic tracking sensor is usually attached to the ultrasound transducer, allowing the position and the orientation of the transducer to be tracked. Ultrasound calibration refers to the procedure for determining the fixed transformation between ultrasound images and the tracking device or sensor attached to the ultrasound transducer.

Accordingly, an improved method and system for overcoming the problems in the art is desired.

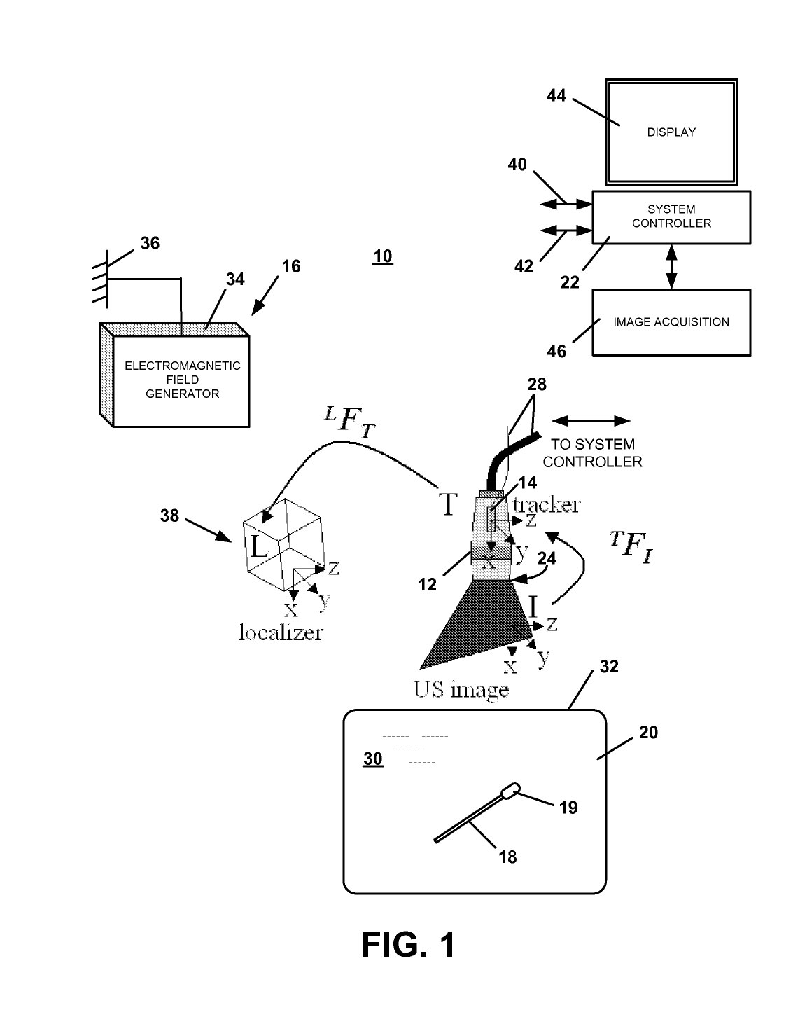

FIG. 1 is a partial block diagram view of a system for automatic calibration of tracked ultrasound according to an embodiment of the present disclosure;

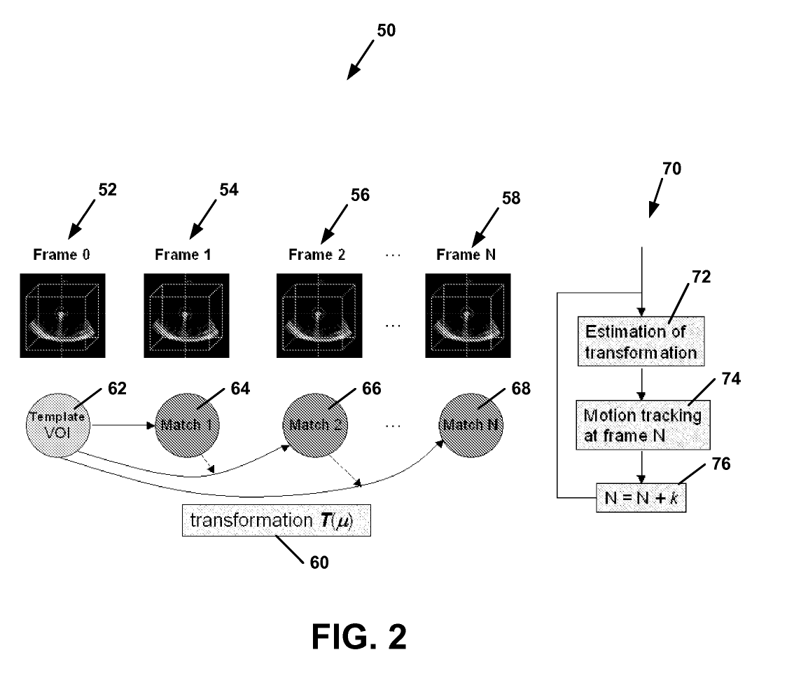

FIG. 2 is a simplified schematic and flow diagram view illustrating a method for automatic calibration of tracked ultrasound according to an embodiment of the present disclosure; and

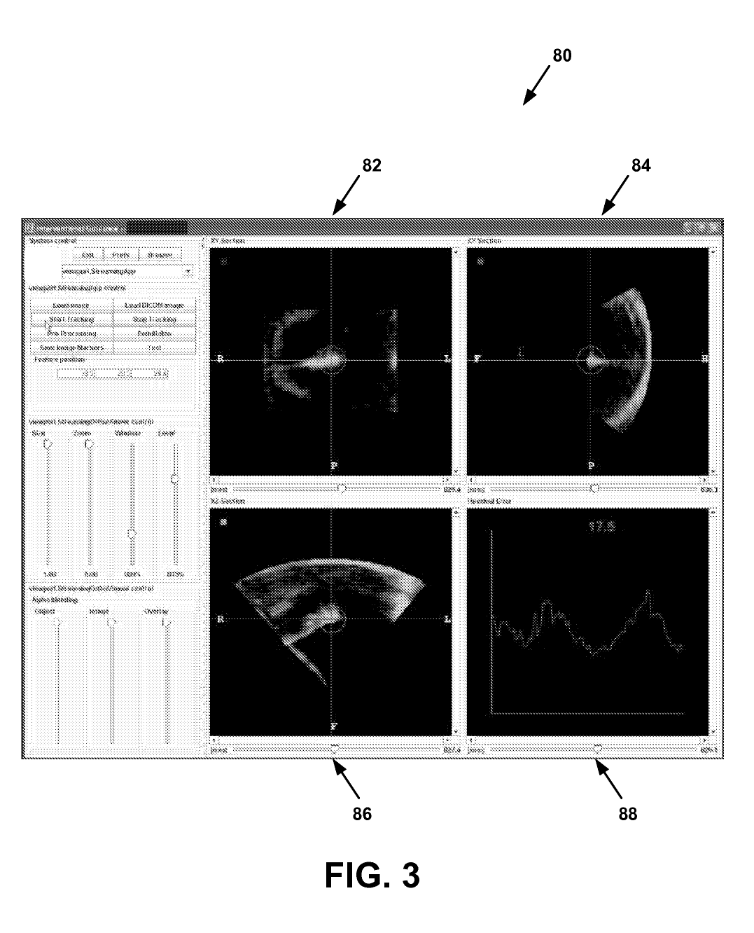

FIG. 3 is a screen display view illustrating three multi-planar reconstruction image views of an ultrasound volume obtained via image-based tracking according to another embodiment of the present disclosure.

In the figures, like reference numerals refer to like elements. In addition, it is to be noted that the figures may not be drawn to scale.

As used herein, calibration of tracked ultrasound means determining the spatial relationship between the live ultrasound image and the attached spatial tracker. In other words, ultrasound calibration refers to the procedure to determine the fixed transformation between the ultrasound images and the attached tracking device. In response to the problems in the art, the embodiments of the present disclosure advantageously provide a method for fully automatic calibration of tracked ultrasound.

FIG. 1 is a partial block diagram view of an ultrasound imaging system 10 for implementing automatic calibration of tracked ultrasound according to an embodiment of the present disclosure. The ultrasound imaging system 10 comprises an ultrasound transducer 12, a tracker 14 coupled to the ultrasound transducer, a localizer (generally indicated by reference numeral 16), a calibration feature 18, a container 20, and a system controller 22. The tracker 14 is coupled to the ultrasound probe 12 in a given position and orientation with respect to an energy emission surface 24 of the ultrasound transducer 12. The ultrasound transducer or probe 12 can include any suitable handheld ultrasound transducer or probe that can be configured for implementing the embodiments of the present disclosure and for carrying out the requirements of a given ultrasound imaging application. Ultrasound probe 12 includes an ultrasound transducer (not shown) located within its housing, proximate surface 24, for emission of desired ultrasound energy in an image field (generally indicated by reference numeral 26). Various electrical power and signal lines, collectively represented by the feature indicated by reference numeral 28, are coupled between the ultrasound probe 12, tracker 14, and system controller 22, for example, as appropriate, for carrying out various functions and steps described herein.

The calibration feature 18 is adapted to be at least partially immersed and moveable within a volume of liquid, gel, or other suitable aqueous medium (as generally represented by reference numeral 30), and at various positions and orientations, according to the requirements of a given tracked ultrasound calibration. The volume of liquid, gel, or other suitable aqueous medium 30 is contained within a suitable container or tank 20. The container or tank 20 includes at least one surface 32 suitable for transmission of ultrasound energy, in response to the ultrasound transducer emitting energy and the surface 24 of ultrasound probe 12 being in contact with surface 32.

The localizer 16 includes a tracking generator 34, the tracking generator 34 being configured to emit tracking energy for use in connection with the tracker 14 and a calibration object 18. In one embodiment, the tracking generator 34 comprises an electromagnetic field generator, wherein the generator is referenced to a fixed orientation and location, as indicated by reference numeral 36. Electromagnetic field generator 34 generates an electromagnetic field in a region of interest, also referred to as a given localizer space or volume of interest, generally indicated by reference numeral 38.

System controller 22 can comprise any suitable computer and/or ultrasound transducer interface, the controller further being programmed with suitable instructions for carrying out the various functions as discussed herein with respect to performing automatic calibration of tracked ultrasound as discussed herein. System controller 22 may include various input/output signal lines, such as 40 and 42, for example, for being electronically coupling to (ii) other elements of the ultrasound imaging system 10 or (ii) one or more remote computer systems outside of ultrasound imaging system 10. A suitable display device 44 is coupled to system controller 22, for example, for use by a system operator during a given automatic calibration of tracked ultrasound. Furthermore, additional devices, such as input/output devices, pointing devices, etc. (not shown) may be provided, as may be necessary, for use in carrying out one or more portions of a given implementation of automatic calibration of tracked ultrasound. In addition, a means 46 for obtaining an image acquisition from storage (e.g., a memory or storage device containing previously obtained images from a given modality) or real-time image acquisition (e.g., real-time images from a given modality acquisition device) is coupled to system controller 46.

According to one embodiment of the present disclosure, the method of ultrasound calibration includes solving as a point-based registration problem in which the ultrasound image coordinates P_{I} of a common point set are registered to their corresponding coordinates P_{L} in a localizer space. FIG. 1 contains illustrations of the various coordinate systems applicable for implementing the automatic calibration of tracked ultrasound as presented herein, including a coordinate system L for the localizer space, a coordinate system T for the tracker space, and a coordinate system I for the ultrasound image space. Further as illustrated in FIG. 1, the overall transformation can be expressed as the multiplication of homogeneous transformation matrices, given by the equation:

P_{L}=^{L}F_{T}·^{T}F_{I}·Pᵢ (1)

where P_{L} and P_{I} are the coordinates of a control point in the coordinate systems of the localizer space and the ultrasound image, respectively. The term ^{L}F_{T} is the real-time tracking result of the localizer for the tracker attached to the ultrasound transducer at the time the control point is identified in the ultrasound image. The term ^{T}F_{L} is the fixed transformation between the tracker and the image. Given a sufficient number of points (N≧3), ^{T}F_{I} can be solved using singular value decomposition (SVD).

However, many traditional ultrasound calibration approaches require human interaction to identify the image coordinates of the control points. The manual procedure is time consuming, which may disadvantageously lead to problems when commercializing the ultrasound guidance system. In addition, accurate ultrasound calibration may need a large number of control points. Therefore, a fully automatic calibration approach is highly desired. In addition, while some recent calibration approaches have realized full automation, such calibration approaches disadvantageously rely on complex phantoms. According to the embodiments of the present disclosure, the method of ultrasound calibration includes using an image processing algorithm integrated with the calibration procedure. As a result, neither human interaction nor additional complex hardware is required to achieve full automation of the ultrasound calibration procedure.

According to one embodiment of the present disclosure, a method for ultrasound calibration includes using an image processing algorithm to localize a number of sets of control points in the ultrasound image space. As a result, an unlimited number of control points can be used for the ultrasound calibration, thereby allowing for high calibration accuracy. In addition, the system for implementing the method of ultrasound calibration is simple and low cost. Furthermore, the ultrasound calibration is fast and carried out automatically (i.e., with no manual determination of control points).

While the embodiments are described herein with respect to 3D ultrasound calibration, the embodiments can be used for 2D ultrasound calibration as well. In addition, according to another embodiment, real-time (2D or 3D) ultrasound images can be streamed (e.g., using suitable video streaming techniques) to a computer separate from the ultrasound diagnostic imaging system for implementing one or more portions of the ultrasound calibration procedure. Furthermore, if the ultrasound images are 2D, then the images can be acquired via frame-grabbing of images contained in a video output signal of the ultrasound scanner or system controller 22 of the ultrasound diagnostic imaging system 10.

According to one embodiment, at least the tip 19 of the tracked needle 18 is submerged in a tank 20 of gel or water 30. A six degrees of freedom (6 DOF) tracker 14 is attached to the ultrasound transducer 12, allowing the position and orientation of the transducer to be tracked by the exterior localizer 16. Similarly, a tracker is provided with the needle 18, for example, using a miniature sensor integrated in the needle tip 19. The needle, in particular, at least the needle tip 19 which includes the miniature sensor, is moved within the tank with respect to the transducer, resulting in a position change of the needle tip in the ultrasound volume. The ultrasound frame of the ultrasound volume is processed after the needle's motion, from a previous position to the new position within the volume, to determine the needle's new image position. In addition, the localizer 16 tracks both (i) the needle and (ii) the tracker of the ultrasound transducer during the procedure.

The method of automatic ultrasound calibration further includes using image registration to identify the needle tip in the ultrasound volume. FIG. 2 illustrates an example of the image processing algorithms 50, each of which matches a template of the needle tip 19 to the current image of the needle tip in the corresponding ultrasound frame (52, 54, 56, and 58). The number of frames comprises N≧3. For an initial frame 52, a template with the volume of interest (VOI) 62 is established. The image processing algorithm proceeds to a next frame 54, and uses image processing to match the template of the needle tip (from frame 52) to the image of frame 54, corresponding to Match 1 indicated by reference numeral 64. The process continues with frame 56, and uses image processing to match the template of the needle tip (from the initial frame 52 and frame 54) to the image of frame 56, corresponding to Match 2 indicated by reference numeral 66. The process continues similarly in this manner, through frame 58, and uses image processing to match the template of the needle tip (from the initial frame 52, frame 54, frame 56 and any additional intervening frames) to the image of frame 58, corresponding to Match N indicated by reference numeral 68.

In addition, the needle's motion can be modeled by a parameterized transformation such as the pure translation, the rigid-body, or the affine transformation, etc. For example, if the needle is moved manually, the rigid-body or affine transformation may be used to account for translation, rotation, and artifact changes of the needle in the ultrasound images. If a robot with three translational joints is available to move the needle, then the translation model should be used to increase the motion tracking algorithm's robustness and accuracy. Assuming the needle's motion is continuous, the motion parameters of one ultrasound frame can be used to estimate the motion of the next frame. A local image registration problem is solved at each individual frame using numerical optimization (e.g., the Gauss-Newton method). The tracking algorithm is fast and can be carried out in real time. The average processing time in one embodiment is on the order of 35 ms (or 28.6 Hz) per frame using a 3.2 GHz workstation with two gigabytes (2G) of random access memory (RAM). Details of one example of a suitable motion tracking algorithm can be found in S. Xu, J. Kruecker, S. Settlemier and B. J. Wood, “Real-time motion tracking using 3D ultrasound” Proc. SPIE Vol. 6509, 65090× (Mar. 21, 2007).

FIG. 2 further illustrates a transformation T(μ) indicated by reference numeral 60 and a simplified flow diagram 70 for establishing an estimation of the transformation (step 72), motion tracking at a given frame N (step 74), incrementing the value of N to a next value N+k, with k≧1 (step 76), and repeating the flow at step 72. The tracking result of frame N is used to estimate the motion of frame N+k, for example, using a suitable motion tracking algorithm. The cycle repeats itself until a desired number of frames have been registered to each other.

FIG. 3 shows a screen shot 80 of one embodiment of software tracking of the needle's tip. The three image views (82, 84, and 86) are the multi-planar reconstruction of the ultrasound volume. View 82 represents a multi-planar reconstruction of the ultrasound volume for an XY-section of a given frame. View 84 represents a multi-planar reconstruction of the ultrasound volume for a ZY-section of the given frame. View 86 represents a multi-planar reconstruction of the ultrasound volume for an XZ-section of the given frame. Using template matching and image processing techniques as discussed herein, the needle tip is automatically identified and displayed in all the three views. With respect to the various image views and in connection with a fixed coordinate system relative to a patient, various designations can be provided with the image views. For example, the designation L can represent left, R can represent right, F can represent feet, H can represent head, etc. Cross-hairs can also be provided to mark a correspondence between different image views or viewing sections, such as commonly used in viewing 3D images. The bottom right view (or window) 88 shows residual error of the template matching at each ultrasound frame (over a series of frames). In view 88 of FIG. 3, the residual error of a current motion tracking was determined to have 17.5 as its value. The value indicates an accuracy of the motion tracking, which can be used in the calibration procedure to avoid including frames with tracking results which occur outside of an acceptable range (i.e., corresponding to inaccurate tracking results).

In addition, using the symbols of equation (1), the needle tip's position is P_{I} for the image-based tracking, and P_{L} for the localizer based tracking. One such point pair can be calculated automatically for each ultrasound image frame, allowing the acquisition of hundreds or thousands of point pairs, which leads to significantly higher accuracy compared to the manual method. With manual identification of P_{I} in each acquired frame, the number of points is typically limited to the range of 10-50. The time savings per point pair is on the order of a factor of 1000, since accurate manual identification of the needle tip in 3D takes on the order of 30-60 seconds.

According to another embodiment, while the ultrasound calibration method has been described with respect to 3D ultrasound calibration, the method is also applicable to 2D ultrasound calibration. With 2D ultrasound calibration, the method further includes confining the needle's motion to the imaging plane of the 2D ultrasound transducer, for example, with use of a needle guide or other suitable means for confining the needle's motion to the imaging plane.

According to another embodiment, the image-based algorithm detects the relative motion between the needle and the transducer. In this embodiment, the needle is in a fixed position and the ultrasound transducer is moved relative to the needle.

According to a further embodiment, any instrument suitable for generating stable features in ultrasound images can be used instead of the needle. In addition, in yet another embodiment, the image-processing algorithm locates the stable feature's position by segmenting the feature in respective ultrasound images. Image segmentation refers to the process of partitioning a digital image into multiple regions (or sets of pixels). Generally, one region corresponds to one object. Examples of suitable instruments can include a sphere, a cube, or anything else that can be accurately localized in ultrasound images.

Furthermore, the image-processing algorithm can track both tissue motion and instrument motion. This embodiments of the present disclosure can be applied to the field of image-guided surgery, particularly, surgical intervention that requires guidance and fusion of ultrasound images.

By now it will be appreciated that a method for automatic calibration of tracked ultrasound comprises configuring a localizer (i) to track a position and orientation of an ultrasound transducer within a localizer space and (ii) to track a position and orientation of calibration feature within the localizer space. An ultrasound volume suitable for transmission of ultrasound is provided, wherein the ultrasound volume is positioned within the localizer space. The calibration feature is disposed in the ultrasound volume and a series of ultrasound images is acquired of the ultrasound volume with the ultrasound probe as a relative positioning and orientation of the ultrasound transducer and calibration feature is varied. The method utilizes image processing to determine an image-based position and orientation of the calibration feature within each frame of the series of ultrasound images. The method further includes computing transformation parameters of the tracked ultrasound calibration as a function of (i) the image-based position and orientation of the calibration feature within the series of ultrasound images, (ii) the corresponding position and orientation of the localizer tracked ultrasound transducer for each selected frame of the series of ultrasound images, and (iii) the corresponding position and orientation of the localizer tracked calibration feature for each frame of the series of ultrasound images, wherein the transformation parameters spatially relate the localizer coordinate space to the ultrasound image space.

According to one embodiment, the localizer is configured (i) to track a position and orientation of the ultrasound transducer within the localizer space and (ii) to track a position and orientation of the calibration feature within the localizer space. In one embodiment, the ultrasound transducer includes a tracker coupled to the transducer, wherein the localizer tracks the tracker within the localizer space. In addition, the calibration feature comprises any instrument suitable for generating at least one stable feature in an ultrasound image. In another embodiment, the calibration feature comprises a needle, wherein the image processing determines the position and orientation of the tip of the needle within each image of the series of ultrasound images.

According to another embodiment, the method utilizes an ultrasound volume suitable for transmission of ultrasound, wherein the ultrasound volume is positioned within the localizer space. The ultrasound volume can include, for example, a tank containing at least one selected from the group consisting of a gel and water.

Disposing the calibration feature in the ultrasound volume can include disposing the calibration feature in the ultrasound volume by moving the calibration feature through the ultrasound volume. In one embodiment, moving the calibration feature includes using a robotic arm having three translational joints to move the calibration feature through the ultrasound volume.

In one embodiment, the step of acquiring a series of ultrasound images of the ultrasound volume with the ultrasound probe as a relative positioning and orientation of the ultrasound transducer and calibration feature is varied, comprises wherein the series of ultrasound images includes N frames, where N is greater than or equal to three (N≧3). In one embodiment, the ultrasound images comprise 3D images. In another embodiment, the ultrasound images comprise 2D images, wherein the method further comprises confining a motion of the calibration feature through the ultrasound volume in an imaging plane of the 2D images. For example, confining the motion an include using a guide that confines the motion to the 2D imaging plane. In yet another embodiment, the relative positioning and orientation of the ultrasound transducer and calibration feature is varied by maintaining the ultrasound transducer stationary while moving the calibration feature through the ultrasound volume.

In another embodiment, utilizing image processing to determine an image-based position and orientation of the calibration feature within each frame of the series of ultrasound images can comprise determining the image-based position and orientation of the calibration feature, for each frame, by processing the ultrasound image to determine the calibration feature's image position and image orientation. The step can further comprise wherein determining the calibration feature's image position and image orientation includes matching a template of the calibration feature to a current image of the calibration feature in each frame of the image of the series of ultrasound images.

In yet another embodiment, computing transformation parameters of the tracked ultrasound calibration comprises computing as a function of (i) the image-based position and orientation of the calibration feature within the series of ultrasound images, (ii) the corresponding position and orientation of the localizer tracked ultrasound transducer for each frame of the series of ultrasound images, and (iii) the corresponding position and orientation of the localizer tracked calibration feature for each frame of the series of ultrasound images, wherein the transformation parameters spatially relate the localizer coordinate space to the ultrasound image space. Computing can include solving for the transformation parameters using singular value decomposition (SVD). In addition, computing can further include automatically calculating a point pair for each ultrasound image frame, the point pair including (i) an image-based tracking point P_{I} of an identifiable portion of the calibration feature and (ii) a localizer-based tracking point P_{L} of the ultrasound transducer. In another embodiment, the calibration feature comprises a needle and the identifiable portion of the needle comprises the needle's tip.

In a still further embodiment, a motion of the calibration feature is configured to be a continuous motion, wherein the method further comprises modeling the motion of the calibration feature by a parameterized transformation. In this embodiment, motion parameters of one ultrasound image frame are used to estimate motion of the calibration feature in a subsequent ultrasound image frame. In addition, modeling the motion includes solving a local image registration problem at each individual ultrasound image frame using numerical optimization.

In addition, the embodiments of the present disclosure include a diagnostic ultrasound imaging system configured to implement automatic calibration of tracked ultrasound according to the methods disclosed herein.

Although only a few exemplary embodiments have been described in detail above, those skilled in the art will readily appreciate that many modifications are possible in the exemplary embodiments without materially departing from the novel teachings and advantages of the embodiments of the present disclosure. For example, the embodiments of the present disclosure can be applied to any ultrasound scanner integrated with a localizer. Accordingly, all such modifications are intended to be included within the scope of the embodiments of the present disclosure as defined in the following claims. In the claims, means-plus-function clauses are intended to cover the structures described herein as performing the recited function and not only structural equivalents, but also equivalent structures.

In addition, any reference signs placed in parentheses in one or more claims shall not be construed as limiting the claims. The word “comprising” and “comprises,” and the like, does not exclude the presence of elements or steps other than those listed in any claim or the specification as a whole. The singular reference of an element does not exclude the plural references of such elements and vice-versa. One or more of the embodiments may be implemented by means of hardware comprising several distinct elements, and/or by means of a suitably programmed computer. In a device claim enumerating several means, several of these means may be embodied by one and the same item of hardware. The mere fact that certain measures are recited in mutually different dependent claims does not indicate that a combination of these measures cannot be used to an advantage.

1. A method for automatic calibration of tracked ultrasound comprising:
configuring a localizer (16) (i) to track a position and orientation of an ultrasound transducer (12) within a localizer space (38) and (ii) to track a position and orientation of calibration feature (18) within the localizer space;
providing an ultrasound volume (20) suitable for transmission of ultrasound, wherein the ultrasound volume is positioned within the localizer space;
disposing the calibration feature (18) in the ultrasound volume (20);
acquiring a series of ultrasound images (52,54,56,58) of the ultrasound volume with the ultrasound probe as a relative positioning and orientation of the ultrasound transducer (12) and calibration feature (18) is varied;
utilizing image processing (62,64,66,68) to determine an image-based position and orientation of the calibration feature within each frame of the series of ultrasound images; and
computing transformation parameters of the tracked ultrasound calibration as a function of (i) the image-based position and orientation of the calibration feature (18) within each of the series of ultrasound images, (ii) the corresponding position and orientation of the localizer tracked ultrasound transducer (12) for each selected frame (52,54,56,58) of the series of ultrasound images, and (iii) the corresponding position and orientation of the localizer tracked calibration feature (18) for each frame of the series of ultrasound images, wherein the transformation parameters spatially relate the localizer coordinate space (L) to the ultrasound image space (I).

2. The method of claim 1, wherein the ultrasound transducer (12) includes a tracker (14) coupled to the transducer, and wherein the localizer tracks the tracker within the localizer space.

3. The method of claim 1, wherein the calibration feature (18) comprises any instrument suitable for generating at least one stable feature in an ultrasound image.

4. The method of claim 3, further wherein the calibration feature (18) comprises a needle, wherein the image processing determines the position and orientation of the tip (19) of the needle within each image of the series of ultrasound images.

5. The method of claim 1, wherein the ultrasound volume (20) includes a tank containing at least one selected from the group consisting of a gel and water.

6. The method of claim 1, wherein disposing the calibration feature (18) in the ultrasound volume (20) includes moving the calibration feature through the ultrasound volume.

7. The method of claim 6, further wherein moving the calibration feature (18) includes using a robotic arm having three translational joints to move the calibration feature through the ultrasound volume.

8. The method of claim 1, wherein the series of ultrasound images includes N frames, where N is greater than or equal to three (N≧3).

9. The method of claim 1, wherein the ultrasound images comprise 3D images.

10. The method of claim 1, wherein the ultrasound images comprise 2D images, the method further comprising:
confining a motion of the calibration feature through the ultrasound volume in an imaging plane of the 2D images.

11. The method of claim 10, further wherein confining the motion includes using a guide that confines the motion to the 2D imaging plane.

12. The method of claim 1, wherein the relative positioning and orientation of the ultrasound transducer (12) and calibration feature (18) is varied by maintaining the ultrasound transducer stationary while moving the calibration feature through the ultrasound volume.

13. The method of claim 1, wherein determining the image-based position and orientation of the calibration feature (18) includes, for each frame (52,54,56,58), processing the ultrasound image to determine the calibration feature's image position and image orientation.

14. The method of claim 1, further wherein determining the calibration feature's image position and image orientation includes matching a template (62,64,66,68) of the calibration feature to a current image of the calibration feature in each frame of the image of the series of ultrasound images.

15. The method of claim 1, wherein computing includes solving for the transformation parameters using singular value decomposition (SVD).

16. The method of claim 1, wherein computing further includes automatically calculating a point pair for each ultrasound image frame, the point pair including (i) an image-based tracking point P_{I} of an identifiable portion of the calibration feature and (ii) a localizer-based tracking point P_{L} of the ultrasound transducer.

17. The method of claim 16, wherein the calibration feature (18) comprises a needle and the identifiable portion of the needle comprises the needle's tip (19).

18. The method of claim 1, wherein a motion of the calibration feature (18) is continuous, the method further comprising:
modeling the motion of the calibration feature by a parameterized transformation, wherein motion parameters of one ultrasound image frame are used to estimate motion of the calibration feature in a subsequent ultrasound image frame.

19. The method of claim 18, wherein modeling the motion includes solving a local image registration problem at each individual ultrasound image frame using numerical optimization.

20. A diagnostic ultrasound imaging system (10) configured to implement automatic calibration of tracked ultrasound according to the method of claim 1.
